(19) United States
(12) Patent Application Publication
(10) Pub. No.: US 2011/0177516 A1
(43) Pub. Date: Jul. 21, 2011

(54) RAPID ANALYTICAL METHOD FOR MIXED BIOLOGICAL SAMPLES

(75) Inventors: Ralf Himmelreich, Langenfeld (DE); Eva Holzer, Heiligenhaus (DE)

(73) Assignee: Qiagen GmbH

(21) Appl. No.: 13/003,408

(22) PCT Filed: Jun. 6, 2009
(86) PCT No.: PCT/EP2009/004078

§ 371 Date: Apr. 8, 2011

(30) Foreign Application Priority Data
Jul. 10, 2008 (DE) 10 2008 032 501.5

(51) Int. Cl. C12Q 1/68

(52) U.S. Cl. 435/6.12; 435/6.1

(57) ABSTRACT

The present invention relates to a rapid method for the amplification of nucleic acids from biological samples which contain a mixture of different cells or a mixture of cells with contaminating components, in which such cells are lysed in a lysis solution (A) and the lysate can be used immediately subsequently for the analysis using a method amplifying nucleic acid.

The present invention relates to a rapid method for the amplification of nucleic acids from biological samples which comprise a mixture of different cells or a mixture of cells with contaminating components, in which such cells are lysed in a lysis solution (A) and the lysate can be used immediately subsequently for the analysis using a method in which nucleic acid is amplified.

There is currently a great demand for rapid analytical methods with the aid of which genetic material can be examined and, if appropriate, assigned to persons. The prerequisite for such an analytical method is that the genetic material, in the form of isolated DNA or RNA, is made available for the analytical method in such a way that the genetic material is provided in an amount capable of being analyzed and in a purity which suffices for being able to analyze the genetic material. Such an analysis is currently usually performed with the aid of a polymerase chain reaction (PCR), by which the genetic material is amplified.

Even the PCR itself is an analytical method, since it is possible, by selecting specific primers, to identify the presence of corresponding sequences on the isolated nucleic acid; on the other hand, the PCR amplificate itself may be the subject of further analyses.

It is desirable to employ an inexpensive, time-saving method for such an analysis since it is frequently the case that larger amounts of different samples have to be processed simultaneously. Under suitable conditions, the polymerase chain reaction is a highly selective and sensitive method; however, when unsuitable conditions or buffer systems are chosen, it may lead to undesired results.

WO 03/102184 A1 describes a method of separating nucleic-acid-containing cells from a mixture of different cells or from cells with contaminants by adding a cell-“aggregating” agent (“flocculating agent”) which is preferably bound to a solid surface, and the subsequent isolation of nucleic acids from the cells obtained.

U.S. Pat. No. 5,523,231 describes the isolation of nucleic acids by the unspecific attachment of the nucleic acids to added small spheres with a large surface (“beads”) by cooling the sample and subsequently detaching the nucleic acid from the beads.

WO 2006/103094 discloses a method of isolating nucleic acids from biological material, an improved nucleic acid yield being obtained by the use of nitrogen-containing compounds in the lysis buffer.

The problem of the present invention was to provide a rapid method for the genetic analysis of biological samples in which cells are present as a mixture with other cells or with contaminants, which method can be carried out in a simple and rapid manner and in which method the genetic material to be examined can be isolated without changing the buffer system and an analytical method such as, for example, a polymerase chain reaction, can be carried out.

This problem is solved by a method as claimed in claim 1 and by a lysis solution (A) as claimed in claim 4, with preferred embodiments being detailed in the dependent claims.

The lysis solution (A) of the present invention is based on a buffer solution with a low salt concentration which comprises (a) at least one nonionic surfactant which aids the lysis of the cell wall of the biological sample to be examined and which dissolves protein aggregates. Substances which are suitable for this purpose are, in principle, any nonionic surfactant which has no adverse effect on the nucleic acids to be examined, with Tween 20, Tergitol, Triton X-100; Brij-58 and Nonidet-P40 being used by preference.

The concentration of the nonionic surfactant in the lysis solution (A) employed is between 0.05 and 5%, preferably between 0.1 and 2%, more preferably between 0.2 and 0.8%.

Component (b) of solution (A) is at least one polymer which acts as binder and thickener. The polymer is employed to prevent an inhibition of the subsequent analytical methods by the unspecific complexing of potential inhibitors. Such polymer-based binders and thickeners are known in expert circles. However, preference within the scope of the invention is given to polyvinylpyrrolidone, polyoxazoline, polyethylene glycol, polyvinyl alcohol; Luvitec (BASF). This polymer is employed in solution (A) in a concentration in the range of from 0.05 to 0.5%, preferably 0.1% to 0.2%.

A further component (c) which can be employed in the lysis solution may be a proteinase; it is preferred to employ a thermophilic proteinase, especially preferably a thermophilic proteinase selected from among proteinase K or subtilisin. It is very especially preferred to use a thermophilic proteinase K in the lysis solution. The proteinase is preferably employed in such an amount that it provides from 0.5 to 10 μunits, preferably from 1.5 to 4 μunits (international units) per milliliter of lysis solution.

Further components of solution (A) is a pH buffer substance at a concentration of 10-20 mM from the group consisting of Tris; MOPS; HEPES; or phosphate. The pH is preferably brought to 7.5 to 11. The pH is especially preferably at 9.0.

In addition, it is possible to employ a chelating agent for divalent cations in order to inactivate nucleinase. These chelating agents are employed in concentrations of 1 mM and are selected from the group consisting of EDTA and EGTA.

In step (i) in the method for analyzing the nucleic acids, the nucleic-acid-containing cells are first enriched from blood. A variety of methods is available to the skilled worker for this purpose, and these methods are in principle known per se in expert circles.

Such suitable methods include for example the lysing or destroying of undesired cells in the mixture, the addition of surfaces onto which the cells to be lysed or the contaminating components absorb or bind, or filtration, sedimentation, selection, or by centrifugation, or a combination of a plurality of these methods, without being limited thereto. The preferred object of this step (i) is to separate the cells which contain genetic material from other cells or contaminating components, or to enrich them with respect to the other components.

Starting materials which may be employed in the method according to the invention are samples which, besides nucleic-acid-containing cells, also comprise other cells or other contaminating components. Examples of such biological samples are whole blood, buffy coats, mixtures of blood and other materials such as, for example, tissue, sperm, lymph, urine, fecal matter or similar, and punched disks from what are known as blood-test cards. The method according to the invention is particularly suitable when it is preferred to separate the cells which harbor genetic material from other cells or from contaminating components.

In a preferred embodiment according to the invention, blood samples are first treated with an erythrocyte lysis buffer (B) so that the erythrocytes in the sample are lysed. Then, the remaining white blood cells are separated from the lysis mixture by centrifugation. The supernatant is discarded and the white blood cells are immediately available for further processing.

The lysis buffer (B) serves exclusively for lysing erythrocytes which are present in blood. Any erythrocyte lysis buffer known in expert circles can be used for this purpose, without limiting the invention thereby. Preferred as a lysis buffer is ELB1 (320 mM sucrose; 50 mM Tris/Cl pH 7.5; 5 mM MgCl₂; 1% Triton X-100) or ELB2 (155 mM NH₄Cl; 10 mM; KHCO₃).

In a further preferred embodiment, the sample is brought into contact with a surface onto which the cells to be lysed adsorb or bind. Thereafter, the remaining constituents of the sample are largely removed by a separation method. To this end, the sample is brought into contact with suitable surfaces onto which the cells absorb or bind. Examples of such surfaces which are suitable for this purpose are small spheres, also known as beads, for example made of glass, silica, polymers or coated beads, preferably magnetic beads. However, it is also possible to modify the surfaces of the consumables such as, for example, reaction vessels, reaction filters, filter columns, spin filter minicolumns, membranes, frits, glass fiber fabric, dishes, tubes, (pipette) tips or wells of multiwall plates for the binding. In an especially preferred embodiment, magnetic beads are used.

For the lysis of erythrocytes, a surface can be introduced in the form of modified magnetic particles. The white blood cells adsorb onto the surfaces and can be enriched by magnetic separation.

Beads which are suitable for the present method may comprise any type of magnetic beads known to date in which a magnetic core is coated with a glass or polymer coating and which on their surface bear groups which make possible an unspecific attachment or binding of nucleic-acid-containing cells onto the beads. It is preferred to employ those beads which are hydrophilic on their surface, for example which bear acid groups, preferably carboxylic acid groups, phosphoric acid groups or sulfuric acid groups, or their salts, more preferably carboxylic acid groups or their salts, it being possible for the abovementioned groups to be bound directly to the surface or to be part of the polymer which forms the surface coating, to be bound to the surface via spacer molecules or to be parts of a compound which are bound to the surface of the beads. In a preferred embodiment, the beads bear on their surface a total charge which is weakly negative in overall terms, since the cells are bound particularly effectively when such conditions prevail. A neutral to weakly-positive charge of the beads is also possible, although not preferred for the present method. Examples of suitable carboxylated polymers which are suitable as coating material for the beads and which provide a surface which is suitable for the invention are described in detail in the German patent application DE 10 2005 040 259.3. Examples of compounds which may be bound to the surface are glycine, hydrazine, aspartic acid, 6-aminocaproic acid, NTA (nitrilotriacetic acid), polyacrylic acid (PAA), glycerin, diglyme (diethylene glycol dimethyl ether), glyme (dimethoxyethane), pentaerythritol, toluene or combinations of these, without being limited thereto. Likewise preferred magnetic beads are those which are described in the German patent application DE 10 2005 058 979.9. Such suitable magnetic beads are commercially available.

Further examples of suitable beads are silica beads, such as, for example, MagAttract Suspension-B, MagAttract Suspension-G (all by Qiagen).

The cells are brought into contact with the magnetic beads over a sufficiently long period of time, i.e. a period of time which suffices to allow the cells to bind/attach themselves to the beads. Such a period of time should be at least 30 s, preferably at least 1 min, further preferably at least 3 min.

After the attachment/binding of the cells to the magnetic particles, the cells can be collected or separated from the medium which surrounds the cells by applying a magnetic field to the vessel in which the cells together with the beads are located. In a preferred embodiment, a magnet is applied externally to the vessel in which the cells and the magnetic beads are located, and the remainder of the sample is removed from the vessel, for example decanted off, or removed with the aid of a suitable device, for example drawn off with the aid of a pipette. The magnetic particles together with the cells can optionally be resuspended in a suitable wash medium and thereby washed, whereafter a magnetic field is again applied to the vessel and the wash medium is again removed from the vessel. The present method therefore provides a particularly gentle handling of the cells.

After the enrichment in step (i) of the nucleic-acid-containing cells to be lysed, these cells are brought into contact in step (ii) with a lysis solution (A) which is described in detail hereinabove. After the cells have been in contact with the lysis solution (A), this lysis mixture is, in step (iii), incubated for a period of from 1 minute to 3 hours, preferably from 2 minutes to 1 hour, more preferably from 5 to 20 min at a temperature of from room temperature to below 100° C., preferably at least 60° C., more preferably at at least 70° C. and especially preferably between 75 and 80° C. Alternatively, a two-step incubation may be performed. Thus, for example, it might be possible to incubate first for 10 min at 56° C. and thereafter for 5 min at 80° C.

After the incubation step (iii), the lysis mixture may, in an optional step (iv), be centrifuged in order to separate insoluble cell components from the nucleic acids; however, such a step is not necessarily required. If as described above magnetic particles are employed in the lysis of the erythrocytes, a magnetic separation may be performed prior to removing the lysate for the subsequent detection reaction, in order to avoid carry-over of magnetic particles.

Beads may remain in the mixture, but should preferably not be carried over into the detection reaction. DNA and RNA is present in the lysate in free form because the lysate does not have any properties which support the binding of nucleic acid to the beads.

In a further step (v), the nucleic acids thus obtained are multiplied by means of a method which amplifies the nucleic acids. Such methods are well known in expert circles and are not limiting for the invention. A preferred example for such a method is a polymerase chain reaction (PCR), if appropriate with a preceding reverse transcription (RT-PCR). The amplification may be performed with specific primers or with unspecific primers. By using specific primers, it is possible to enhance either one or more specific gene sequence(s) from the genetic material, or, when using specific primers according to international standard, a genetic fingerprint may be generated. In addition, it is also possible to use unspecific primers, when comparing a known sample with an unknown sample in order to find out about the origin of the unknown sample.

The components used for the method may be provided in the form of a kit, which simplifies carrying out the method. Such a kit comprises at least the lysis solution (A) or its starting materials, that is components (a), (b) and (c) and, if appropriate, the buffer with the low salt concentration, and instructions as to how and in what ratios the above must be combined, and, preferably, either a lysis buffer (B) or a solid surface onto which either the cells to be lysed or contaminating components adsorb or bind. Furthermore, the kit may comprise components which are suitable for carrying out a PCR.

An advantage of the present method is that genetic material may, in few steps, be obtained from a cell mixture or a mixture of contaminating components with cells which harbor genetic material, in a quality such that it can be employed directly in an amplification method. This permits the simultaneous treatment of a large sample quantity of different samples for examining the genetic material present therein, for example in forensics, in the identification of persons or in the assignment of samples which contain genetic material to persons from which this material has been obtained.

A further advantage of the method described herein is that contamination of the samples can be kept to a minimum, due to the fact that only few processing steps are carried out, since in particular the overall processing of the samples is performed in the same vessels so that transferring of the samples to be analyzed is dispensed with.

### FIGURES

FIG. 1

Results of an H18 Real Time PCR: the data shown are the averaged C(t) values of in each case four independent preparations with a variant of solution (A).

FIG. 2

β-Actin Real Time PCR results of two different donor blood samples (donor 1 and 2) with different anti-coagulants, numbered from 1-6, in comparison with QIAamp Blood Mini-prepped controls (only carried out with anticoagulant 1; referred to as “1 QA”). Key to the anticoagulants/blood collection systems: 1) Sarstedt (Monovette) EDTA KE/9 ml; 2) Sarstedt (Monovette) Li-heparin LH/7.5 ml; 3) Sarstedt (Monovette) Coagulation 9 NC/10 ml; 4) B-D Vacutainer K2E 18 mg/10 ml; 5) B-D Vacutainer 9NC 0.105M/9 ml; 6) B-D Vacutainer K3E 15% 0.12 ml/10 ml

### EXAMPLES

### Example 01

### Blood Protocol in the Two-Step Method with Centrifugation

Blood is treated with an erythrocyte lysis buffer, whereby the red blood cells lyse immediately. White blood cells or their nuclei and/or mitochondria are precipitated by means of centrifugation. After the sediment have been washed, they are lysed by means of solution (A). The lysate can be employed directly in PCR reactions.

When carrying out the above in practice, 200 μl of blood together with 600 μl of erythrocyte lysis buffer (106 mM sucrose, 16 mM Tris/Cl pH 7.5, 1.6 mM MgCl₂ 0.33% Triton X-100) are placed into a 1.5 ml reaction microvessel. After the vessel has been closed, it is inverted 5 times in order to mix the liquids. The vessel is then centrifuged for 20 seconds at 10 000×g and the supernatant is discarded: The sediment is resuspended in 400 μl of wash buffer (80 mM sucrose, 12.5 mM Tris/Cl pH 7.5, 1.25 mM MgCl₂ 0.25% Triton X-100) and again precipitated by centrifugation (20 sec at 10 000×g). The sediment is then resuspended in solution (A) and incubated for 5 min at 80° C. and 1400 rpm in an Eppendorf Thermomixer. The lysate is ready and can be employed directly in a PCR.

### Composition of Solution (A)

| A1 | A2 | A3 | G7 |
|---|---|---|---|
| 0.1% PVP | 0.1% PVP | 0.1% PVP | — |
| — | 0.45% Tween-20 | 0.45% Tween-20 | 0.2% Tween 20 |
| 0.45% NP-40 | — | 0.45% NP-40 | 1 mM EGTA |
| 10 mM Tris | 10 mM Tris pH 7.5 | — | 1 mM EDTA |
| pH 7.5 | | | |
| 1 mM EDTA | 1 mM EDTA | 1 mM EDTA | 20 mM Tris |
| | | | pH 10.5 |

The solutions are complemented by the addition of 0.5 μl proteinase K per 200 μl buffer

### Quantitative PCR (Specific for the Human H18 Gene)

The H18-specific real-time PCR was carried out with 12.5 μl QIAGEN QuantiTect Probe PCR Mastermix, 0.10 μl of H18 forward primer (100 μM), 0.10 μl of H18 reverse (100 μM), 0.05 μl of H18 probe (100 μM), 8.75 μl of double-distilled water and 4.0 μl of lysate aliquot. The temperature cycles were 15 minutes at 95° C. and 40×(15 seconds at 95° C., 1 minute at 60° C.). The amplification was carried out in each case with eluates from 4 separate nucleic acid preparations.

### Result

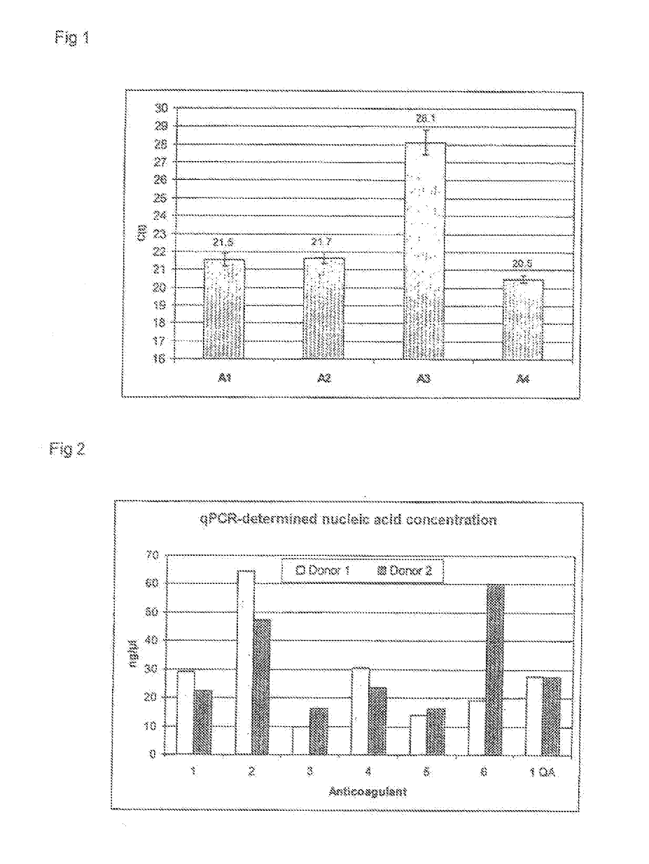

The results of the real-time PCR in FIG. 1 demonstrate clearly that the method works very well. One aspect regarding the composition of solution (A) appears to be the maintenance of the pH range. In the case of an unbuffered solution (A) variant (A3), the result deteriorates markedly, while at pH 10.5 (A4) the result improves markedly.

### Example 02

### Blood Protocol in the Two-Step Method with MagBeads

Blood is treated with an erythrocyte lysis buffer which simultaneously contains magnetic particles with carboxylated surfaces. The red blood cells lyse immediately, and the white blood cells or their nuclei and/or mitochondria bind to the magnetic particles and are enriched by means of magnetic separation. After the cells/cell fractions which are bound to the magnetic particles have been washed, they are lysed by means of solution (A). After magnetic separation, the lysate can be employed directly in PCR reactions. The QIAamp Blood Mini Protocol (QIAGEN GmbH; Hilden) was performed as specified in the manual, by way of comparative procedure.

When carrying out the above in practice, 200 μl of blood together with 600 μl of erythrocyte lysis buffer (106 mM sucrose, 16 mM Tris/Cl pH 7.5, 1.6 mM MgCl₂ 0.33% Triton X-100) and 60 μl of carboxylated magnetic particles (50 mg/ml) are placed into a 1.5 ml reaction microvessel. The vessel is then left to shake for 2 min at 1400 rpm on an Eppendorf Thermomixer. The tube is placed into a magnetic separation rack, and the supernatant is discarded once the magnetic particles have separated completely. After the addition of 500 μl of wash buffer (80 mM sucrose, 12.5 mM Tris/Cl pH 7.5, 1.25 mM MgCl₂ 0.25% Triton X-100), the magnetic sediment is resuspended by vortexing briefly, and, again, the supernatant is discarded after the magnetic separation.

Then, the sediment is resuspended in 75 μl of solution (A) (0.1% PVP, 0.45% Tween-20, 0.45% NP-40, 10 mM Tris/Cl pH 7.5, 1 mM EDTA) and 0.5 μl of proteinase K and incubated for 5 min at 80° C. and 1400 rpm in an Eppendorf Thermomixer. The lysate is ready and can be employed directly in a PCR. It is preferred that the tube is placed in a magnetic separation rack before the withdrawal in order to avoid a transfer of magnetic particles into the PCR.

### Quantitative PCR (Specific for the β-Actin Gene)

The β-actin-specific real-time PCR was carried out with 12.5 μl of QIAGEN QuantiTect Probe PCR Mastermix, 0.10 μl of β-actin forward primer (100 μM), 0.10 μl of β-actin reverse primer (100 μM), 0.03 μl of β-actin probe (100 μM), 8.75 μl of double-distilled water and 4.0 μl of lysate aliquot. The temperature cycles were 15 minutes at 95° C. and 40×(15 seconds at 95° C., 1 minute at 60° C.). The amplification was carried out in each case with eluates from 4 separate nucleic acid preparations.

### Results

The concentration of the DNA prepared is comparable to the QIAamp-purified samples. The variations of the concentration determined are caused by the blood donors and the blood collection tube with the different anticoagulants employed and can be considered to be “normal”. Apart from the time advantage of the procedure, the method proposed within the context of the invention gives results which are comparable to established reference methods.

1. A method of analyzing nucleic acids from a biological sample which contain a mixture of different cells or a mixture of cells and contaminating components, with cells being present which contain genetic material, characterized in that
(i) nucleic-acid-containing cells are enriched
(ii) the cells of step (i) is brought into contact with a lysis solution (A) which comprises at least the following components: (a) at least one nonionic surfactant, (b) at least one polymer which acts as a binder and thickener;
(iii) the biological sample is incubated in the lysis solution to form a lysis mixture;
(iv) if appropriate, the lysis mixture is centrifuged;
(v) a method which amplifies the nucleic acids is carried out in immediate succession,
without a purification of the nucleic acids between step (ii) and step (v) being necessary.

2. The method of claim 1, wherein the lysis solution (A) additionally comprises (c) a proteinase.

3. The method of claim 1, wherein the lysis solution (A) additionally comprises (d) a chelating agent for divalent cations.

4. The method of claim 1, wherein the lysis solution (A) additionally comprises (e) a buffer substance, so that the pH is between 7.5 and 10.5.

5. The method of claim 1, wherein the incubation in step (iii) is carried out least 60° C., preferably at least 70° C. and especially preferably at between 75° C. and 80° C.

6. A lysis solution (A) for lysing cells of a biological sample, comprising (a) at least one nonionic surfactant, (b) at least one polymer which acts as a binder, and (c) at least one proteinase, preferably a thermophilic proteinase.

7. The lysis solution of claim 6, wherein component (a) in solution (A) is selected from the group consisting of Tween, Tergitol, Triton X 100, Nonidet, P40, and Brij58.

8. The lysis solution of claim 6, wherein component (b) in solution (A) is selected from the group consisting of polyvinylpyrrolidone, polyoxazoline, polyethylene glycol, polyvinyl alcohol, and Luvitec.

9. The lysis solution of claim 6, wherein the proteinase (c) in solution (A) is proteinase K.

10. The method of claim 1, wherein in step (i), the nucleic-acid-containing cells are enriched by: lysing or destroying undesired cells; adding surfaces onto which the nucleic-acid-containing cells or the contaminating components adsorb or bind; centrifugation; filtration; sedimentation; dissecting; selecting or a combination of a plurality of these methods.

11. The method of claim 1, wherein the biological sample is whole blood, buffy coats, a mixture of blood and urine, fecal matter, lymph or tissue or a punched disk from a blood test card, and in that the nucleic-acid-containing cells which are enriched in step (i) are white blood cells.

12. The method of claim 1, wherein the amplification of the nucleic acids in step (v) is performed by PCR or RT-PCR.

13. A kit for carrying out the method of claim 1, comprising at least one lysis solution (A) or its components (a), and (b).

14. The kit of claim 13, additionally comprising a lysis buffer (B) or a solid surface onto which either the cells to be lysed or contaminating components adsorb or bind.
